(19) United States
(12) Patent Application Publication
(10) Pub. No.: US 2025/0017458 A1
(43) Pub. Date: Jan. 16, 2025

(54) REMOVABLE ENDOSCOPE CAP INCLUDING A FILTER FOR IMPROVED STONE FREE RATE

(71) Applicant: Lumenis, Ltd., Yokneam Ilit (IL)

(72) Inventor: Vitaly Rondel, Hadera, IL (US)

(73) Assignee: Lumenis, Ltd., Yokneam Ilit (IL)

(21) Appl. No.: 18/770,171

(22) Filed: Jul. 11, 2024

Related U.S. Application Data
(60) Provisional application No. 63/513,264, filed on Jul. 12, 2023.

(51) Int. Cl. A61B 1/06; A61B 1/00; A61B 1/07; A61B 1/307; A61B 18/00; A61B 18/26

(52) U.S. Cl. CPC A61B 1/0646; A61B 1/00137; A61B 1/0676; A61B 1/07; A61B 1/307; A61B 18/26; A61B 2018/00511

(57) ABSTRACT

A removable cap for an endoscope used with a surgical laser to improve the stone free rate (SFR) of lithotripsy procedures. The removable cap includes a filter configured to block or filter light of a wavelength like a wavelength of light from an aiming beam generated by the surgical laser.

### CROSS-REFERENCE TO RELATED APPLICATIONS

This application claims the benefit of U.S. Provisional Patent Application Ser. No. 63/513,264 filed on Jul. 12, 2023, the disclosure of which is incorporated herein by reference.

### TECHNICAL FIELD

The present disclosure generally relates to endoscopes and particularly, but not exclusively, to improving stone visibility and/or identification with endoscope cameras when used in conjunction with a surgical laser system.

### BACKGROUND

Endoscopes are used to obtain an internal view of a patient. To that end, endoscopes have one or more cameras. The purpose of the camera is to provide an image of the target (e.g., organ, stone, or the like) on which the medical procedure is being performed or an image in the vicinity of where the medical procedure is to be performed. For example, an endoscope can be used to view the interior of a kidney and/or assess kidney stones in the kidney. As another example, an endoscope can be used to view a ureter possibly having stones, tumors, or strictures. In yet another example, an endoscope can be used to view a bladder and its anatomy such as the ureter openings and possible treatment targets like stones or tumors.

Lithotripsy is a frequent treatment where surgical laser systems are used with an endoscope. In such a treatment, energy from the laser is directed at stones in the urinary system (e.g., kidney, bladder, ureter, or the like) to break up and/or disintegrate the stones. At the conclusion of the treatment, a medical practitioner will visually scan the urinary system for any remaining (e.g., missed, or the like) urinary stones or fragments of stones to ensure a stone free rate (SFR). However, it can be difficult to identify stones, particularly small stones, surrounded by tissue when using the endoscope for visual examination.

Thus, there is a need to improve identification or imaging of stones to improve the SFR of lithotripsy procedures.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides a removable cap for an endoscope used with a surgical laser to improve the stone free rate (SFR) of lithotripsy procedures. The removable cap includes a filter configured to block or filter light of a wavelength like a wavelength of light from an aiming beam generated by the surgical laser.

Some embodiments of the disclosure can be implemented as a removable cap for an endoscope. The removable cap can comprise a housing having an open proximal end and a distal end, the open proximal end defining an inner cavity in the housing, the inner cavity dimensioned to fit over a distal end of an endoscope; and a filter disposed on the distal end of the housing, the filter to align with a camera of the endoscope when the housing is inserted over a distal end of the endoscope.

In further embodiments of the removable cap, the inner cavity is shaped to fit an outer shape of the distal end of the endoscope.

In further embodiments, the removable cap can comprise an aperture in the distal end of the housing, the aperture to align with a working channel of the endoscope when the housing is inserted over the distal end of the endoscope.

In further embodiments of the removable cap, the aperture allows an optical fiber inserted through the working channel to be extended out of the distal end of the endoscope a working distance.

In further embodiments of the removable cap, the aperture can provide that one or more backlights disposed in the distal end of the endoscope are not covered by the housing when the housing is disposed over the distal end of the endoscope.

In further embodiments, the removable cap can comprise one or more optically transparent portions disposed in the distal end of the housing, the one or more optically transparent portions to align with one or more backlights disposed in the distal end of the endoscope when the housing is disposed over the distal end of the endoscope.

In further embodiments of the removable cap, the filter is a long pass filter (LPF).

In further embodiments of the removable cap, the filter is a band pass filter (BPF).

In further embodiments of the removable cap, the LPF is configured to block or filter light having a wavelength below 550 nanometers (nm).

With some embodiments, the disclosure can be implemented as an endoscopic system. The endoscopic system can comprise an endoscope comprising a camera assembly disposed in a distal end of the endoscope and at least one working channel in which an optical fiber can be inserted; and a removable cap for the endoscope. The removable cap can comprise a housing having an open proximal end and a distal end, the open proximal end defining an inner cavity in the housing, the inner cavity dimensioned to fit over the distal end of the endoscope; and a filter disposed in the distal end of the housing, the filter to align with the camera assembly when the housing is inserted over a distal end of the endoscope.

In further embodiments of the endoscopic system, the removable cap can comprise an aperture in the distal end of the housing, the aperture to align with a working channel of the endoscope when the housing is inserted over the distal end of the endoscope.

In further embodiments of the endoscopic system the aperture can allow an optical fiber inserted through the working channel to be extended out of the distal end of the endoscope a working distance.

In further embodiments of the endoscopic system the endoscope can comprise one or more backlights, the removable cap comprising one or more optically transparent portions disposed in the distal end of the housing, the one or more optically transparent portions to align with one or more backlights disposed in the distal end of the endoscope when the housing is disposed over the distal end of the endoscope.

In further embodiments of the endoscopic system, the filter is a long pass filter (LPF) or a band pass filter (BPF).

In further embodiments of the endoscopic system, the LPF or the bandpass filter is configured to block or filter light having a wavelength below 550 nanometers (nm).

Some embodiments of the disclosure can be implemented as a method. The method can be for a lithotripsy procedure or to improve an SFR of a lithotripsy procedure. The method can comprise installing a removable cap over the distal end of an endoscope, the endoscope comprising a camera and the removable cap comprising a filter, wherein the filter optically aligns with the camera; positioning the distal end of the endoscope with the removable cap in an environment treated via a lithotripsy procedure; activating and aiming beam of a lasing console coupled to an optical fiber disposed in a working channel of the endoscope; and passing the optical fiber over a treatment area comprising tissue and capture images with the endoscope camera.

In further embodiments, the method can comprise verifying a stone free rate (SFR) based on the images captured with the endoscope camera.

In further embodiments of the method, the removable cap can comprise a housing having an open proximal end and a distal end, the open proximal end defining an inner cavity in the housing, the inner cavity dimensioned to fit over a distal end of an endoscope; and a filter disposed on the distal end of the housing, the filter to align with a camera of the endoscope when the housing is inserted over a distal end of the endoscope.

In further embodiments of the method, the filter is a long pass filter (LPF) or a band pass filter (BPF).

In further embodiments of the method, the LPF or the bandpass filter is configured to block or filter light having a wavelength below 550 nanometers (nm).

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A, FIG. 1B, and FIG. 1C illustrate an endoscope system including a removable cap with a filter in accordance with at least one embodiment of the present disclosure.

FIG. 2A, FIG. 2B, and FIG. 2C illustrate a lithotripsy system in accordance with at least one embodiment of the present disclosure.

FIG. 2D and FIG. 2E illustrate the lithotripsy system of FIG. 2A to FIG. 2C during a lithotripsy procedure in accordance with at least one embodiment of the present disclosure.

FIG. 3 illustrates an image captured by an endoscope.

FIG. 4A and FIG. 4B illustrate images captured by an endoscope system without a removable cap with a filter disposed over the distal end of the endoscope accordance with at least one embodiment of the present disclosure.

FIG. 5A and FIG. 5B illustrate images captured by an endoscope system with a removable cap with a filter disposed over the distal end of the endoscope accordance with at least one embodiment of the present disclosure.

FIG. 6 illustrates a method to improve a stone free rate (SFR) of a lithotripsy procedure in accordance with at least one embodiment of the present disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

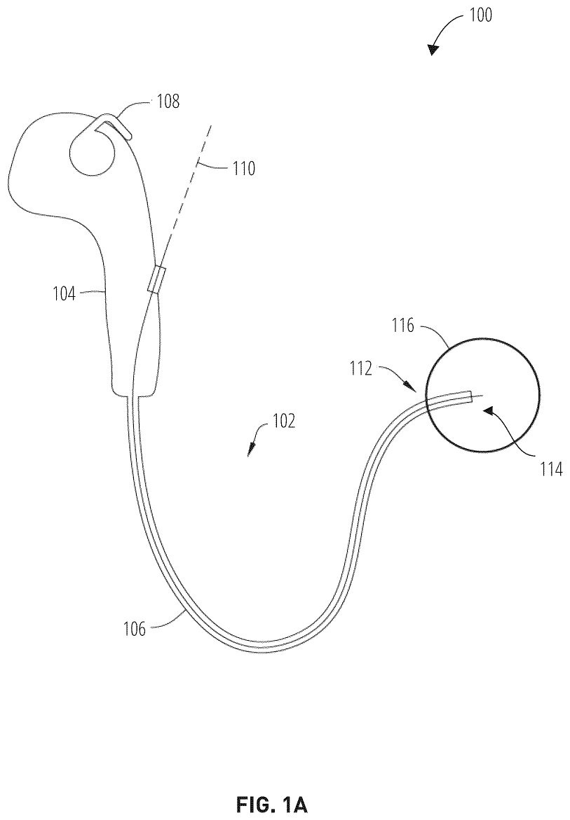

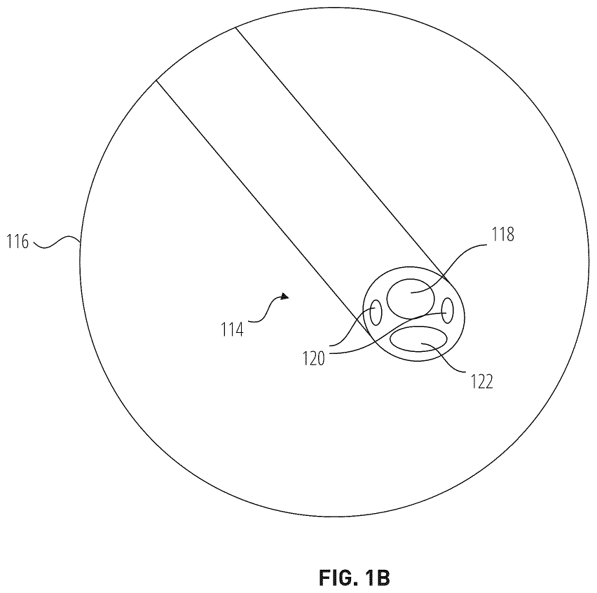

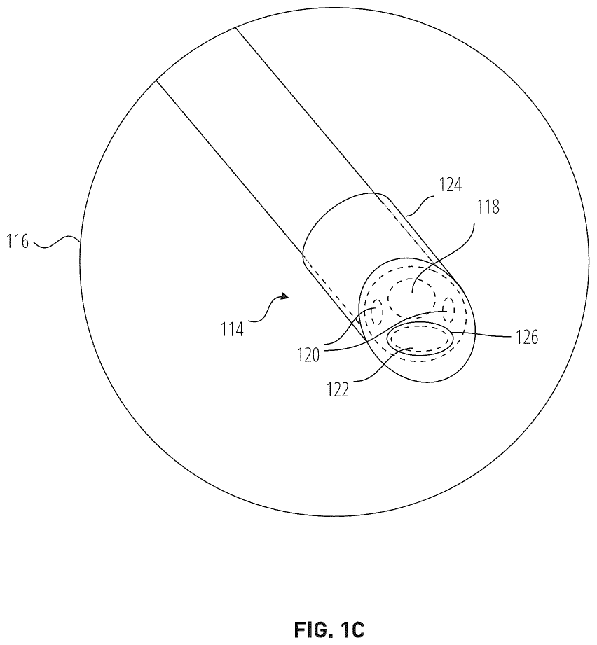

FIG. 1A illustrates an endoscope system 100 with an integrated filter on the camera, according to various examples provided herein. As depicted, the endoscope system 100 includes an endoscope 102 having a handle 104 and catheter 106. The handle 104 has an actuator 108 (described in greater detail below) while the catheter 106 has at least one working channel in which an optical fiber 110 can be inserted. At the distal end 112 of the catheter 106 is an endoscope tip 114. The endoscope tip 114 is depicted in greater detail by enlarged view 116 shown in FIG. 1B. Although the disclosure is described with reference to lithotripsy and ureteroscope, endoscope 102 can be any of a variety of “scopes” such as, for example, a ureteroscope, a colonoscope, a bronchoscope, or the like.

As can be seen from enlarged view 116 in FIG. 1B, the distal end 112 of catheter 106 includes endoscope tip 114. Endoscope tip 114 includes a working channel aperture 118 (wherein a working length of optical fiber 110 can be extended to emit laser energy during a lithotripsy procedure. Further, endoscope tip 114 includes backlight 120 and endoscope camera assembly 122. Backlights 120 can be any of a variety of light emitting elements, such as, for example, light emitting diodes (LEDs), optical waveguides, or the like.

In general, endoscope camera assembly 122 can be any of a variety of camera assemblies including a sensor and/or a lens. Endoscope camera assembly 122 can be configured to capture images which can be used as the basis for a video feed generated by endoscope 102. Although often endoscope camera assembly 122 will be disposed in the distal end 112 of the endoscope 102, some embodiments may provide an optical waveguide optically coupling the distal end 112 of the endoscope 102 to endoscope camera assembly 122 positioned in another location of the endoscope (e.g., the proximal end, or the like).

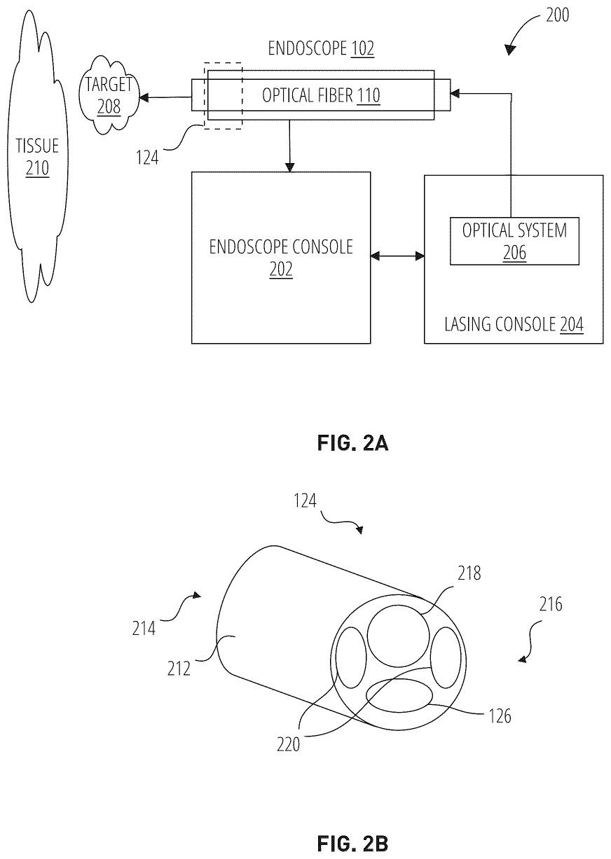

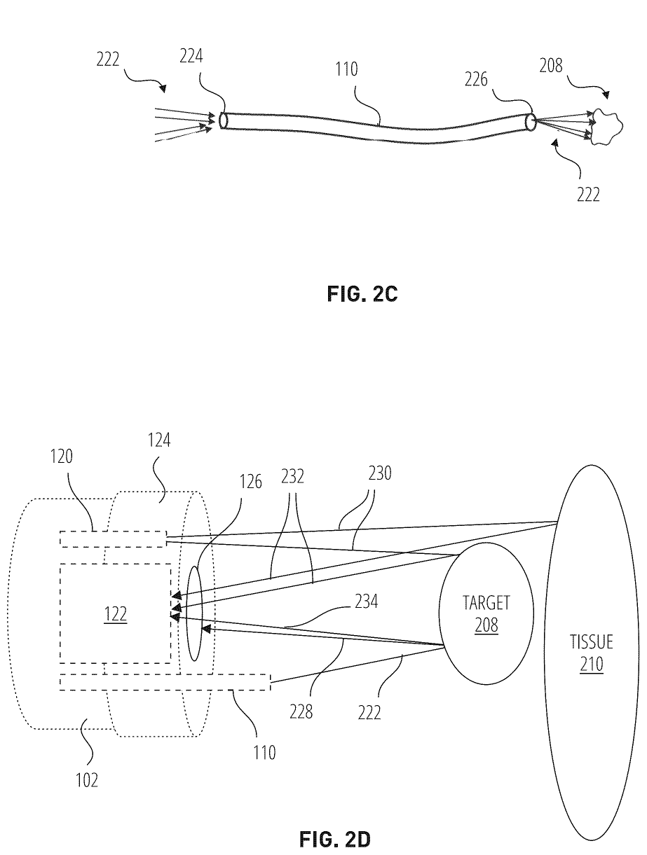

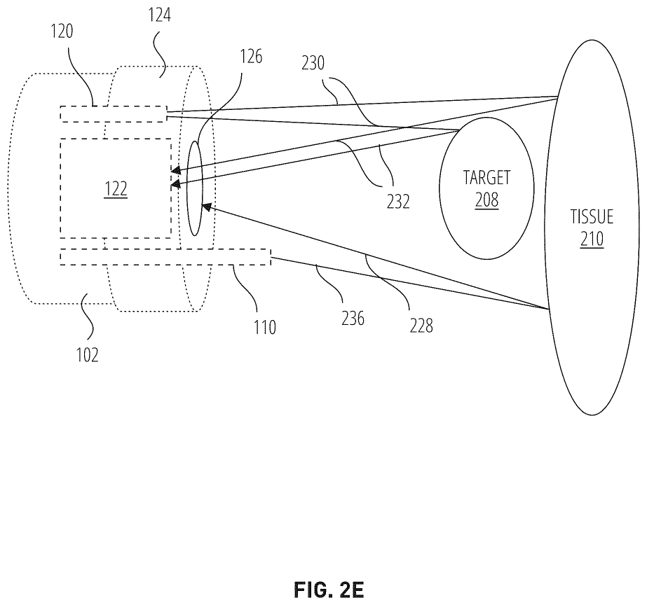

Endoscope system 100 can further include a removable cap 124 with a filter 126. As shown in FIG. 1C, the removable cap 124 can be placed over the distal end 112 of catheter 106 and particularly over endoscope tip 114. The filter 126 can be a short pass filter (SPF), a band pass filter (BPF), or a long pass filter (LPF) configured to filter or block certain wavelengths of light and pass or transmit other wavelengths of light. The filter 126 is position on the removable cap 124 such that when the removable cap 124 is placed over the endoscope tip 114, the filter 126 covers or optically alights with the endoscope camera assembly 122. Other portions of the removable cap 124 can be transmissive to all light wavelengths or to wavelengths associated with backlights 120. Further, in some embodiments, removable cap 124 can include an aperture (see FIG. 2B) aligned with working channel aperture 118 such that optical fiber 110 can be extended out from working channel aperture 118 and extend out of removable cap 124. In such a manner, light from optical fiber 110 and backlight 120 can be transmitted freely but light incident on endoscope camera assembly 122 can be filtered by filter 126.

For example, filter 126 can be a LPF configured to filter or block wavelengths of light below a cutoff wavelength and transmit wavelengths of light equal to or above the cutoff wavelength. In some embodiments, the cutoff wavelength can be between 450 nanometers (nm) and 650 nm or between 380 nm and 650 nm. In a specific embodiment, the cutoff wavelength can be 550 nm. In such an example, the filter 126 will be configured to block or filter light with wavelengths below 550 nm (including light from an aiming beam of the surgical laser system, described in greater detail below) and pass or transmit light with wavelengths equal to or above 550 nm (including light from the backlights 120 and fluorescence light emitted by a stone responsive to incidence of the aiming beam light). This is described in greater detail below.

FIG. 2A show an exemplary lithotripsy system 200 configured to provide imaging or stone visualization to improve SFR, in accordance with example of the present disclosure. Lithotripsy system 200 can comprise an endoscope console 202 and a lasing console 204. The endoscope console 202 and lasing console 204 can be coupled to the endoscope 102 and the optical fiber 110, respectively. As noted above, the endoscope 102 can include one or more working channels in which the optical fiber 110 can be inserted.

Lasing console 204 includes optical system 206, which in general includes one or more laser light sources and various optics arranged to generate a laser beam. Typically, optical system 206 will include at least two light sources; one arranged to generate an aiming beam to identify a target 208 and ensure that the optical fiber 110 is pointed at the target 208, and a second to generate a treatment beam to therapeutically treat (e.g., ablate, dust, etc.) the target 208. Such laser sources can include, but are not limited to, solid-state lasers, gas lasers, diode lasers, and fiber lasers. Further, optical system 206 includes optics to shape and/or couple the generated laser beam(s) to optical fiber 110.

The various optics within optical system 206 can include, but are not limited to, one or more polarizers, beam splitters, beam combiners, light detector, wavelength division multiplexers, collimators, circulators, and/or lenses. The laser light sources of optical system 206 can comprise a Thulium fiber laser, a Holmium laser, or other types of laser light sources.

Additionally, although endoscope console 202 and lasing console 204 are depicted as separate consoles in FIG. 2A, it is to be appreciated that an embodiment could be implemented where endoscope console 202 and lasing console 204 are combined into a single console, for example, sharing many computing components (e.g., processor, memory, display, controls, etc.). However, for purposes of clarity, the disclosure describes examples where endoscope console 202 and lasing console 204 are separate.

Lithotripsy system 200 further includes removable cap 124 that can be placed over the distal end 112 of endoscope 102 to provide improved imaging and visualization of stones to improve the SFR. As described above, the removable cap 124 includes filter 126, which is configured to selectively block light to improve visualization of the target 208 from the tissue 210 to improve the SFR. Further, endoscope 102 includes endoscope camera assembly 122, with which an image of a target 208 (e.g., stone, tissue, etc.) can be acquired. In some embodiments, the target 208 may be a tissue, a stone, a tumor, a cyst, and the like, within a subject, which is to be treated, ablated, or destroyed. In some embodiments, the subject may be a human being or an animal.

When removable cap 124 is placed over the endoscope tip 114, filter 126 is disposed over endoscope camera assembly 122 (see FIG. 1C and FIG. 2D) and provides that some light is filtered such that images captures by endoscope camera assembly 122 are based on the remaining light (e.g., light not blocked or filtered by filter 126). With some embodiments, for example, as shown in FIG. 2B, removable cap 124 can include aperture 218 aligned with working channel aperture 118 such that optical fiber 110 can be extended out for the distal end 112 of the endoscope 102, through aperture 218, a working distance. Further, removable cap 124 can include optical transparent sections 220 aligned with backlights 120 such that light emitted by backlights 120 will not be blocked by removable cap 124.

As depicted in this figure, removable cap 124 can have a housing 212 having an open proximal end 214 with an inner cavity (FIG. 1C) and a distal end 216. The open proximal end 214 and the inner cavity can be shaped to slide over and attach to the distal end of an endoscope (e.g., distal end 112 of endoscope 102, or the like).

As noted above, lasing console 204 can comprise the optical system 206, which is arranged to generate a laser beam. FIG. 2C illustrates a portion of the lithotripsy system 200 including laser light 222 associated with the generated laser beam (e.g., the aiming beam, a treatment beam, etc.). The laser light 222 can be directed by optical fiber 110 to target 208 or tissue 210. For example, the optical fiber 110 comprises a proximal end 224 and a distal end 226. The proximal end 224 is coupled to optical system 206 and configured to receive laser light 222 while the distal end 226 is the end of the optical fiber 110 extended out of endoscope 102 and from which laser light 222 is emitted. For example, this figure depicts laser light 222 entering the optical fiber 110 at the proximal end 224, propagating through length of the optical fiber 110, exiting the optical fiber 110 at the distal end 226, and being incident on the target 208 from the distal end 226 of the optical fiber 110.

With some embodiments, the aiming beam can be generated with a solid-state laser, a diode laser, or any variety of laser source configured to generate light in the visible spectrum. As a specific example, optical system 206 can be configured to generate an aiming beam having a wavelength of 532 nm, approximately 532 nm, or between 400 nm and 700 nm. The optical system 206 can be further be configured to generate the aiming beam having a power between 2.5 and 30 milliwatts (mW). During and/or at the conclusion of a lithotripsy procedure, the physician may cause lasing console 204 to generate (e.g., via optical system 206) the aiming beam to form incident laser light 236 and scan the optical fiber 110 across an environment to identify a stone or stones to ablate with the treatment beam. Said differently, optical fiber 110 can be scanned across the treatment environment to allow the physician to distinguish between stones (e.g., targets 208) and non-targeted structures (e.g., tissue 210).

As will be appreciated, some amount of laser light 222 will be reflected by the object on which it is incident (e.g., target 208, tissue 210, etc.). When laser light 222 corresponds to the aiming beam, the reflected light will be captured by endoscope camera assembly 122 as the light is in the visible spectrum. In such instances, the reflected laser light may saturate the image. In some instances, the reflected laser light will hide the fluorescence as the reflected laser light has a higher magnitude or higher brightness that will obscure the fluorescence, thereby obscuring any indication of whether the image is a targeted object (e.g., target 208) or a non-targeted object (e.g., tissue 210). Accordingly, it may be difficult to determine whether all targets 208 (e.g., stones) have been ablated, dusted, or otherwise destroyed ensuring a high SFR.

This is illustrated more fully in FIG. 2D and FIG. 2E. As depicted in FIG. 2D, when laser light 222 is incident on target 208, some of laser light 222 will be reflected by target 208 as reflected laser light 228. This figure further illustrates accent light 230 generated by backlights 120, which is incident on both target 208 and tissue 210 and generates reflected accent light 232.

It is to be appreciated that urinary stones (e.g., target 208) react to light, especially light in the range of UV to visible (e.g., green aiming beam light generated by optical system 206, or the like) and will emit fluorescence 234 in response to laser light 222 being incident on target 208. However, urinary tissue (e.g., tissue 210) does not react to such light and will not generate fluorescence 234 in response to incidence of laser light 222. It is noted that the wavelength of fluorescence 234 is longer than the wavelength of laser light 222 and reflected laser light 228. For example, laser light 222 having a wavelength of approximately 532 nm will be excite fluorescence 234 when incident on target 208 (e.g., a urinary stone) having a longer wavelength (e.g., 550 nm and longer).

Filter 126 of removable cap 124 can be configured to block or filter reflected laser light 228 but not fluorescence 234 such that images captured by endoscope camera assembly 122 will not be saturated by reflected laser light 228 but will still capture indications of fluorescence 234. Further, reflected accent light 232 can be transmitted by filter (e.g., not blocked) such that the captured images will have an appropriate amount of light to illuminate the scene.

In some embodiments, the wavelength and/or power of aiming beam and thus, laser light 222 can be selected to excite fluorescence 234 from target 208. Further, the filter 126 can be configured to block reflected laser light 228 but not fluorescence 234 and reflected accent light 232.

As noted above, urinary tissue (e.g., tissue 210) does not react to visible light in the same manner that urinary stones do. That is, tissue 210 fluorescence 234 will not be excited from tissue 210 in the same manner that fluorescence 234 is excited from target 208. FIG. 2E illustrates laser light 222 incident on tissue 210. In such a scenario, some of laser light 222 will be reflected by tissue 210 as reflected laser light 228. Further, accent light 230 generated by backlights 120 can be incident on both target 208 and tissue 210 and generate reflected accent light 232.

Filter 126 can block, or filter reflected laser light 228 while allowing reflected accent light 232 to be transmitted. However, as fluorescence 234 is not excited from tissue 210 when laser light 222 is incident on tissue 210, no fluorescence 234 is generated in the example shown in FIG. 2E. As such, the images captured will not depict fluorescence 234 or reflected laser light 228.

It is noted that the present disclosure describes using an aiming beam generated by the optical system 206 to improve the SFR. However, a surgical laser system (e.g., lasing console 204) could be provided with laser sources to generate an aiming beam, a treatment beam, and another beam (e.g., SFR beam) to improve the SFR as outlined herein. As an alternative example, endoscope 102 could be provided with a laser source (e.g., diode laser, solid state laser, or the like) arranged to generate the SFR beam. In such examples, the aiming beam and the SFR beam can have different wavelengths. As a specific example, the aiming beam can have a wavelength in the visible range and be a “green” laser beam while the SFR beam can have a wavelength in the UV range and be a “violet” laser beam.

However, for purposes of clarity, laser systems are described as using the aiming beam for both “aiming” the therapeutic laser and scanning the environment to improve the SFR as outlined herein.

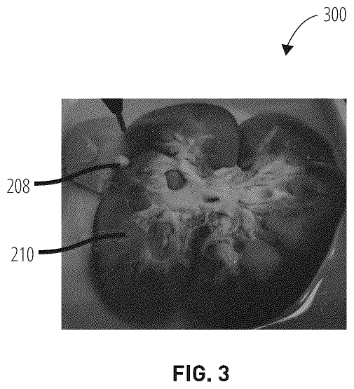

As noted above endoscope camera assembly 122 can be configured to capture an image or image frames of a video stream comprising an environment (or scene) to be treated. FIG. 3 illustrates an endoscopic image 300, which could be captured by endoscope camera assembly 122 of endoscope 102. As depicted in this figure, endoscopic image 300 depicts target 208 and tissue 210. It is noted that the tissue 210 is a porcine kidney and target 208 is a urinary stone.

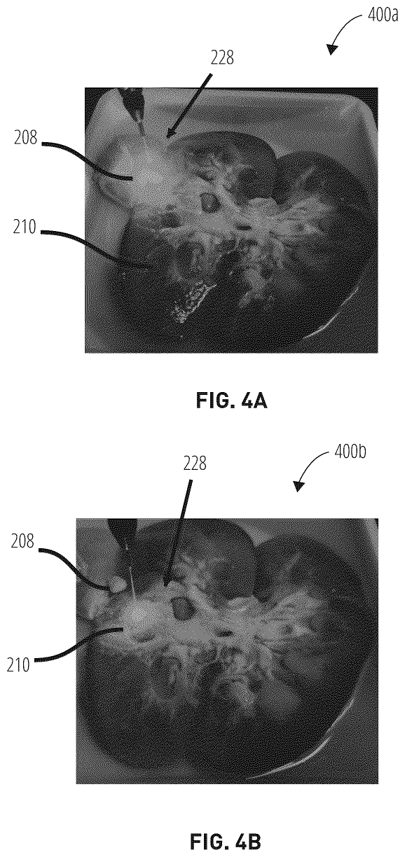

FIG. 4A and FIG. 4B illustrate example endoscopic images captured while laser light 222 corresponding to an aiming beam having a 532 nm wavelength is directed at the target 208 or 210 and while removable cap 124 is not disposed on the distal end of an endoscope. FIG. 4A illustrates endoscopic image 400a, which could be captured by endoscope camera assembly 122 of endoscope 102 when removable cap 124 is not disposed over the distal end 112 of endoscope 102. Endoscopic image 400a shows target 208 and tissue 210 where laser light 222 (not called out) is directed to and incidence on target 208. As can be seen from this figure, the captured image is saturated by reflected laser light 228.

FIG. 4B illustrates an endoscopic image 400b, which could be captured by endoscope camera assembly 122 of endoscope 102 when removable cap 124 is not disposed over the distal end 112 of endoscope 102. Endoscopic image 400b shows target 208 and tissue 210 where laser light 222 (not called out) is directed to and incidence on tissue 210. As can be seen from this figure, the captured image is also saturated by reflected laser light 228.

Accordingly, regardless of whether aiming beam (e.g., laser light 222) is directed to, and be incident on target 208 or tissue 210 it can be difficult to distinguish between a urinary stone and urinary tissue as reflections from the aiming beam saturate the camera sensor.

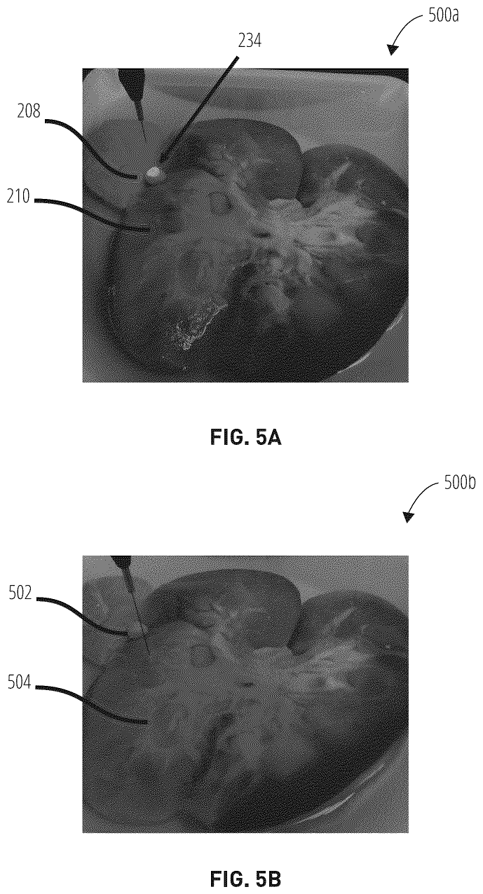

FIG. 5A and FIG. 5B illustrate example endoscopic images captured while laser light 222 corresponding to an aiming beam having a 532 nm wavelength is directed at the target 208 or 210 and while removable cap 124 is disposed on the distal end of an endoscope. FIG. 5A illustrates endoscopic image 500a, which could be captured by endoscope camera assembly 122 of endoscope 102 when removable cap 124 is disposed over the distal end 112 of endoscope 102. Endoscopic image 500a shows target 208 and tissue 210 where laser light 222 (not called out) is directed to and incidence on target 208. Further, endoscopic image 500a depicts fluorescence 234, which is excited by target 208 responsive to laser light 222. However, as filter 126 is configured to block reflected laser light 228, reflected laser light 228 is notably absent from the captured image.

FIG. 5B illustrates an endoscopic image 500b, which could be captured by endoscope camera assembly 122 of endoscope 102 when removable cap 124 is disposed over the distal end 112 of endoscope 102. Endoscopic image 500b shows target 208 and tissue 210 where laser light 222 (not called out) is directed to and be incident on tissue 210. As can be seen from this figure, the captured endoscopic image 500b, like the endoscopic image 500a, is not saturated by reflected laser light 228. Further, as tissue 210 does not excite fluorescence 234 responsive to incidence of laser light 222, fluorescence 234 is also not present in endoscopic image 500b.

Accordingly, endoscopic images 500a and 500b illustrate how removable cap 124 can provide for images captured by endoscope camera assembly 122 of endoscope 102 to differentiate between target 208 and tissue 210, providing a clear indication of whether urinary stones remain and improving the SFR of lithotripsy procedures.

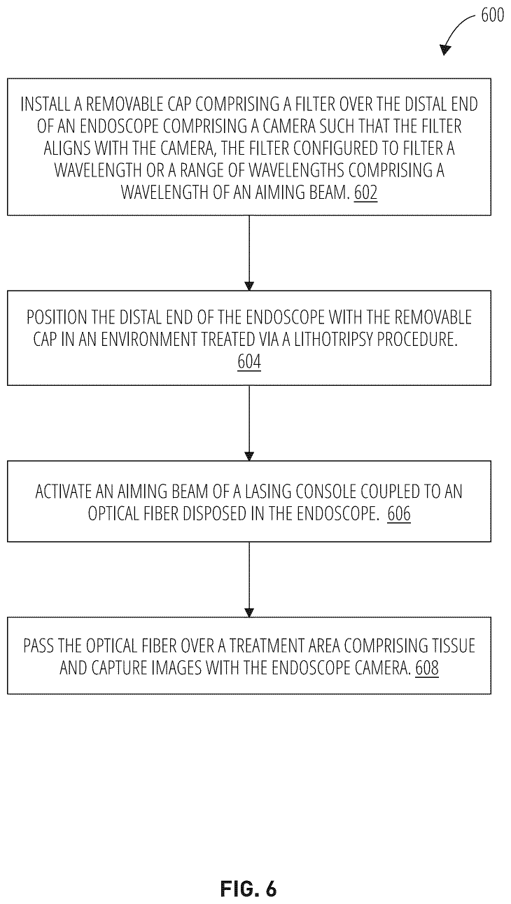

FIG. 6 illustrates a method 600 to improve the SFR of a lithotripsy procedure. Method 600 can be implemented by a physician at the end of a lithotripsy procedure. Method 600 can begin at block 602. At block 602 “install a removable cap comprising a filter over the distal end of an endoscope comprising a camera such that the filter aligns with the camera, the filter configured to filter a wavelength or a range of wavelengths comprising a wavelength of an aiming beam” a removable cap having a filter can be installed on a distal end of an endoscope. For example, removable cap 124 having filter 126 can be installed over the distal end 112 of endoscope 102 such that filter 126 aligns with endoscope camera assembly 122 of endoscope 102.

Continuing to block 604 “position the distal end of the endoscope with the removable cap in a treatment area” the distal end of the endoscope with the removable cap can be positioned in a treatment area (e.g., an environment treated as part of a lithotripsy procedure, or the like). For example, distal end 112 of endoscope 102 can be positioned in an environment including target 208 and/or tissue 210.

Continuing to block 606 “activate an aiming beam of a lasing console coupled to an optical fiber disposed in the endoscope” an aiming beam of a laser source coupled to an optical fiber inserted into the endoscope can be activated. For example, optical system 206 can be activated to generate laser light 222 having a wavelength and power associated with an aiming beam.

Continuing to block 608 “pass the optical fiber over a treatment area comprising tissue and capture images with the endoscope camera” the optical fiber can be passed over the treatment area and particularly the tissue in the treatment area to identify any stones remaining after the lithotripsy treatment to improve the SFR. For example, the distal ends 226 of optical fiber 110 can be passed over tissue 210 while images (e.g., video frames, or the like) are captured by endoscope camera assembly 122 to identify any ones or portions of target 208 that might remain after a lithotripsy procedure. As detailed herein, the stones (e.g., target 208) will be identifiable by the fluorescence 234 represented in images captured by endoscope camera assembly 122 when filter 126 of removable cap 124 is placed over the distal end 112 of endoscope 102 and filter 126 overlap or aligns with endoscope camera assembly 122.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

By using genuine models of anatomy more accurate surgical plans may be developed than through statistical modeling.

What is claimed is:

1. A removable cap for an endoscope, comprising:
a housing having an open proximal end and a distal end, the open proximal end defining an inner cavity in the housing, the inner cavity dimensioned to fit over a distal end of an endoscope; and
a filter disposed on the distal end of the housing, the filter to align with a camera of the endoscope when the housing is inserted over a distal end of the endoscope.

2. The removable cap of claim 1, the inner cavity shaped to fit an outer shape of the distal end of the endoscope.

3. The removable cap of claim 1, comprising an aperture in the distal end of the housing, the aperture to align with a working channel of the endoscope when the housing is inserted over the distal end of the endoscope.

4. The removable cap of claim 3, the aperture to allow an optical fiber inserted through the working channel to be extended a working distance out of the distal end of the endoscope.

5. The removable cap of claim 4, the aperture to provide one or more backlights disposed in the distal end of the endoscope are not covered by the housing when the housing is disposed over the distal end of the endoscope.

6. The removable cap of claim 3, comprising one or more optically transparent portions disposed in the distal end of the housing, the one or more optically transparent portions to align with one or more backlights disposed in the distal end of the endoscope when the housing is disposed over the distal end of the endoscope.

7. The removable cap of claim 1, wherein the filter is a long pass filter (LPF).

8. The removable cap of claim 1, wherein the filter is a band pass filter (BPF).

9. The removable cap of claim 8, wherein the LPF is configured to block or filter light having a wavelength below 550 nanometers (nm).

10. An endoscopic system, comprising:
an endoscope comprising a camera assembly disposed in a distal end of the endoscope and at least one working channel in which an optical fiber can be inserted; and
a removable cap for the endoscope, the removable cap comprising:
a housing having an open proximal end and a distal end, the open proximal end defining an inner cavity in the housing, the inner cavity dimensioned to fit over the distal end of the endoscope; and
a filter disposed in the distal end of the housing, the filter to align with the camera assembly when the housing is inserted over a distal end of the endoscope.

11. The endoscopic system of claim 10, the removable cap comprising an aperture in the distal end of the housing, the aperture to align with a working channel of the endoscope when the housing is inserted over the distal end of the endoscope.

12. The endoscopic system of claim 11, the aperture to allow an optical fiber inserted through the working channel to be extended a working distance out of the distal end of the endoscope.

13. The endoscopic system of claim 12, the endoscope comprising one or more backlights, the removable cap comprising one or more optically transparent portions disposed in the distal end of the housing, the one or more optically transparent portions to align with one or more backlights disposed in the distal end of the endoscope when the housing is disposed over the distal end of the endoscope.

14. The endoscopic system of claim 10, wherein the filter is a long pass filter (LPF) or a band pass filter (BPF).

15. The endoscopic system of claim 14, wherein the LPF or the bandpass filter is configured to block or filter light having a wavelength below 550 nanometers (nm).

31. A method for a lithotripsy procedure, comprising:
installing a removable cap over the distal end of an endoscope, the endoscope comprising a camera and the removable cap comprising a filter, wherein the filter optically aligns with the camera;
positioning the distal end of the endoscope with the removable cap in an environment treated via a lithotripsy procedure;
activating and aiming beam of a lasing console coupled to an optical fiber disposed in a working channel of the endoscope; and
passing the optical fiber over a treatment area comprising tissue and capture images with the endoscope camera.

17. The method of claim 16, further comprising verifying a stone free rate (SFR) based on the images captured with the endoscope camera.

18. The method of claim 16, the removable cap comprising:
a housing having an open proximal end and a distal end, the open proximal end defining an inner cavity in the housing, the inner cavity dimensioned to fit over a distal end of an endoscope; and
a filter disposed on the distal end of the housing, the filter to align with a camera of the endoscope when the housing is inserted over a distal end of the endoscope.

19. The method of claim 16, wherein the filter is a long pass filter (LPF) or a band pass filter (BPF).

20. The method of claim 19, wherein the LPF or the bandpass filter is configured to block or filter light having a wavelength below 550 nanometers (nm).
